(19) United States
(12) Patent Application Publication
(10) Pub. No.: US 2016/0256412 A1
(43) Pub. Date: Sep. 8, 2016

(54) METHOD TO SOLUBILIZE CURCUMINOIDS IN WATER

(71) Applicants: Muhammed Majeed, Edison, NJ (US); Kalyanam Nagabhushanam, East Windsor, NJ (US)

(72) Inventors: Muhammed Majeed, Edison, NJ (US); Kalyanam Nagabhushanam, East Windsor, NJ (US)

(21) Appl. No.: 15/055,766

(22) Filed: Feb. 29, 2016

Related U.S. Application Data
(60) Provisional application No. 62/127,889, filed on Mar. 4, 2015.

(51) Int. Cl. A61K 31/12; A61K 47/20; A61K 9/08; A61K 47/32

(52) U.S. Cl. CPC A61K 31/12; A61K 47/32; A61K 47/20; A61K 9/08

(57) ABSTRACT

A method of solubilizing curcuminoids is disclosed wherein the method uses polyvinyl pyrrolidone and sodium bis (2-ethylhexyl) sulfosuccinate and the aqueous solubility of curcumin/curcuminoid mixtures is enhanced to more than 10%-15% w/v in water. The present invention also pertains to a curcuminoids composition characterised by not less than 10% w/v, more specifically between 10%-15% w/v aqueous solubility of curcuminoids in water.

### CROSS-REFERENCE TO RELATED PATENT APPLICATIONS

This is a non-provisional patent application claiming priority from U.S. Provisional Patent Application No. 62127889 filed on 4 Mar. 2015.

### BACKGROUND OF THE INVENTION

1. Field of the Invention

The invention in general pertains to improved methods for solubilising curcuminoids in water. More specifically, the present invention pertains to a method of solubilising curcuminoids using a combination of polyvinyl pyrrolidone (solubilising agent) and dioctyl sulfosuccinate (surfactant) where previously unreported 10%-15% w/v aqueous solubility of curcuminoids in water is achieved. The present invention also pertains to a curcuminoids composition characterised by 10%-15% w/v aqueous solubility of curcuminoids in water.

2. Description of Prior Art

Curcuminoids, generally extracted from Curcuma longa, have a wide variety of biological activities. Their use has been studied in the treatment of various types of cancers, in neurological diseases such as Alzheimer's, in improving arthritic conditions, for effecting favorable changes in blood lipid profile, as an excellent anti-inflammatory and anti-oxidant and in several other applications. This is despite the fact that curcumin is highly insoluble in water. Curcumin is soluble in the range of 0.432 microgram per ml in purified water at 37° C. (Kumavat Suresh et al., Int. Res. J. Pharm., 2013, 4(5) DOI: 10.7897/2230-8407.04548). There other references those quote a similar figure for this aqueous solubility. This approximately translates to approximately 0.0005% aqueous solubility. United States Patent Application 20140303258 by Majeed et al discuss a method of solubilising curcuminoids using triterpene glycosides wherein a form of soluble curcuminoids as an opaque or turbid emulsion engulfed by triterpene glycosides is visualised. Increasing the solubility of curcumin in water may increase its bioavailability and hence its pharmacological efficacy. Water-soluble curcumin/curcuminoid powders are especially useful for beverage applications. But increasing the solubility from its normal 0.0005% to a desirable level of 10% would require methods that would result in the solubility-enhancement in water to the tune nearly 2000 times. At the present level of technology and science, there are no known methods to solubilize curcumin to the level of 10% or more. Also use of excipients to enhance solubility should result in powders that are safe and stable. The present invention describes increasing aqueous solubility of curcumin/curcuminoid mixtures to more than 10%-15% w/v in water. The inventors used PVP K-30 as a solubilizing agent. PVP K-30 is one such example of polyvinyl pyrrolidone (PVP). Other grades of PVP can be efficaciously used. While PVP is known to be a solubilizing agent, the inventors used selective surfactants that modulate the surface tension of water to enable increased solubilization of curcumin in water. These surfactants include, among others, Dioctyl sodium sulfosuccinate (also known as sodium his (2-ethylhexyl) sulfosuccinate). Also other salts, such as potassium salts of selective surfactants can be used. The invention in particular describes minimizing the quantity of PVP normally used in solubilizing water-insoluble material in aqueous milieu through the use of surfactants. The invention is extendible to other nutraceuticals, pharmaceutical drugs and pesticide aqueous formulations.

It is the principle objective of the present invention to solubilise curcuminoids using PVP and sodium bis (2-ethylhexyl) sulfosuccinate whereby aqueous solubility of curcumin/curcuminoid mixtures to more than 10%-15% w/v in water is achieved. It is another objective of the present invention to disclose a curcuminoids composition characterised by 10%-15% w/v aqueous solubility of curcuminoids in water.

The present invention fulfils the aforesaid objectives and provides further related advantages.

### SUMMARY OF THE INVENTION

Disclosed is a method of solubilizing curcuminoids using PVP and sodium bis (2-ethylhexyl) sulfosuccinate whereby aqueous solubility of curcumin/curcuminoid mixtures to more than 10%-15% w/v in water is achieved. The present invention also discloses a curcuminoids composition characterised by 10%-15% w/v aqueous solubility of curcuminoids in water.

### DETAILED DESCRIPTION OF THE MOST PREFERRED EMBODIMENT

In the most preferred embodiment, the present invention relates to a method of solubilising curcumin/curcuminoid mixtures whereby aqueous solubility of curcumin/curcuminoid mixtures is enhanced to more than 10%-15% w/v in water using polyvinyl pyrrolidone (PVP) and dioctyl sulfosuccinate (AOT) or Docusate sodium, said method comprising steps of:
a) Stirring continuously at room temperature a mixture of polyvinyl pyrrolidone (PVP) and dioctyl sulfosuccinate (AOT) taken in ethanol till the solution of said mixture becomes clear;
b) Adding curcuminoids at room temperature to the solution of step a followed by continuous stirring for 15 hours to further get a clear solution;
c) Evaporating the ethanol in solution of step b using Rota evaporator and further adding distilled water to get a clear mixture;
d) Removing any insoluble material from the clear mixture of step c by filtration; and
e) Spray drying the resulting mixture of step d at 140-150° C. using a spray dryer to get the final product of solubilised curcuminoids wherein the assay of curcuminoids the final product, by HPLC indicates not less than 10% w/w of curcuminoids.

In another most preferred embodiment, the present invention relates to a curcuminoids composition characterised by not less than 10% w/v aqueous solubility of curcuminoids water. In specific embodiment, the present invention relates to a curcuminoids composition characterised by 10%-15% w/v aqueous solubility of curcuminoids in water.

The aforesaid most preferred embodiments are further illustrated as a working embodiments in the following illustrative example.

### Illustrative Example

### Method to Solubilise Curcumin/Curcuminoid Mixtures

Polyvinyl pyrrolidone K30 (60 g) and dioctyl sulfosuccinate (AOT) (2.50 taken in ethanol (500 ml) were stirred continuously at room temperature till the solution of said mixture (mixture solution 1) became dear. Curcuma longa extract (CURCUMIN C3 COMPLEX wherein the chemical assay of said extract defined the content of total curcuminoids by HPLC as not less than 95.0% w/w and not more than 102.0% w/w on dry basis was obtained from Sami Labs Limited, Bangalore, India. 10 grams of aforesaid curcuminoids extract was added to the mixture solution 1 and continuously stirred for is hours at room temperature to get mixture solution 2. Ethanol was evaporated from mixture solution 2 using Rota Evaporator to get mixture solution 3. 350 ml of distilled water was added to mixture solution 3 and filtered to remove insoluble materials to form mixture solution 4. Mixture solution 4 was further spray dried at 140-150° C. using spray dryer. The final product of solubilised curcuminoids (orange-yellowish fine powder) obtained by aforesaid process wherein the aqueous solubility of curcumin/curcuminoid mixtures was raised to more than 10%-15% w/v in water was collected and stored in polyethylene covers. The yield of the final product was 60 grams. It is to be noted that solubility of curcuminoids may be understood as a physical state where curcuminoids go into solution in a manner that upon filtration of said solution no solids (curcuminoid particles) are retained in the interposing medium used in filtration.

The specification established for water soluble curcuminoids was as follows.
1. Content of curcuminoids by HPLC: Not less than 10% w/w
2. Loss on Drying: <10%
3. Solubility: Soluble in water

While the invention has been described with reference to a preferred embodiment, it is to be clearly understood by those skilled in the art that the invention is not limited thereto. Rather, the scope of the invention is to be interpreted only in conjunction with the appended claims.

We claim:

1. A method of solubilising curcumin/curcuminoid mixtures whereby aqueous solubility of curcumin/curcuminoid mixtures is enhanced to more than 10%-15% w/v in water using polyvinyl pyrrolidone (PVP) and dioctyl sulfosuccinate (AOT) or Docusate sodium, said method comprising steps of
a) Stirring continuously at room temperature a mixture of polyvinyl pyrrolidone (PVP) and dioctyl sulfosuccinate (AOT) taken in ethanol till the solution of said mixture becomes clear;
b) Adding curcuminoids at room temperature to the solution of step a followed by continuous stirring for 15 hours to further get a clear solution;
c) Evaporating the ethanol in solution of step b using Rota evaporator and further adding distilled water to get a clear mixture;
d) Removing any insoluble material from the clear mixture of step c by filtration; and
e) Spray drying the resulting mixture of step d at 140-150° C. using a spray dryer to get the final product of solubilised curcuminoids wherein the assay of curcuminoids in the final product by HPLC indicates not less than 10% why of curcuminoids.

2. A curcuminoids composition characterised by not less than 10% w/v aqueous solubility of curcuminoids in water.

3. The composition according to claim 2 characterised by 10%-15% w/v aqueous solubility of curcuminoids in water.
